(19) United States
(12) Patent Application Publication
(10) Pub. No.: US 2013/0229626 A1
(43) Pub. Date: Sep. 5, 2013

(54) Microscope Having A Switchable Documentation Beam Path

(71) Applicant: LEICA MICROSYSTEMS (SCHWEIZ) AG, Heerburgg (CH)

(72) Inventors: René ZUEND, Widnau (CH); Urban Wenk, Eschlikon (CH)

(73) Assignee: LEICA MICROSYSTEMS (SCHWEIZ) AG, Heerbrugg (CH)

(21) Appl. No.: 13/778,612

(22) Filed: Feb. 27, 2013

(30) Foreign Application Priority Data
Mar. 1, 2012 (DE) 10 2012 203 266.5

(51) Int. Cl. A61B 3/13; G02B 21/22; G02B 21/18

(52) U.S. Cl. CPC A61B 3/13; G02B 21/18; G02B 21/22
USPC 351/221; 359/374; 359/376

(57) ABSTRACT

A microscope (1) having a principal observer's beam path (2a) and an assistant's beam path (3a, 3b), and having an optical beam splitter device (5, 15) for generating a documentation beam path (4), where the beam splitter device (5, 15) in a first position outcouples the documentation beam path from the principal observer's beam path (2a), and in a second position outcouples it from the assistant's beam path (3b).

### CROSS REFERENCE TO RELATED APPLICATIONS

This application claims priority of German patent application number 10 2012 203 266.5 filed Mar. 1, 2012, the entire disclosure of which is incorporated by reference herein.

### FIELD OF THE INVENTION

The present invention relates to a microscope having a principal observer's beam path and an assistant's beam path, and having an optical beam splitter device for generating a switchable documentation beam path.

### BACKGROUND OF THE INVENTION

The principal field of application of the present invention that is to be considered hereinafter is surgical microscopy, in particular ophthalmic surgical microscopy, with no intention of thereby limiting the invention to that field. In microsurgical procedures on the anterior segment of the eye, for example cataract operations, illumination light diffusely reflected from the retina, which appears as a reddish transmitted light to a surgeon (principal observer) who is viewing the lens of a patient's eye, is used to make transparent structures in the anterior segment of the eye visible. This type of illumination is referred to as “red reflex” illumination. To implement this illumination type, an illumination beam path proceeding close to the axis with reference to the main optical axis of the microscope extending through the patient's eye is made available. Optimally, the axis of the close-to-axis illumination beam path encloses an angle in the range from 0° to 2° with the main axis of the objective. The illumination beam path can be guided through the main objective, but also past the main objective, to the object region.

An optimum red reflex illumination is less necessary for microsurgical procedures on the posterior segment of the eye; what is instead established is a rather off-axis illumination beam path, for optimum contrast of the observed image.

EP 1 997 423 A1 discloses an ophthalmic surgical microscope which is suitable for making transparent structures in the anterior segment of a patient's eye visible with good contrast. The corresponding surgical microscope contains an illumination device for generating an illumination beam path for close-to-axis illumination, as well as a further illumination beam path for off-axis illumination of the object region. By means of a semi-transparent mirror arranged in the observation beam path, the illumination beam path for close-to-axis illumination is superimposed coaxially and close to the axis onto the observation beam path. By means of a further deflection element, such as a mirror, the illumination beam path for off-axis illumination can be deflected at a greater angle with respect to the optical axis of the observation beam path. In this document, this off-axis illumination is then guided through a field diaphragm of the illumination device and imaged through an illumination optic into an image plane which lies in the object region and is located close to but not exactly in the object plane of the surgical microscope. This results in a delimitation of the illuminated field in the object plane that transitions not abruptly, but instead softly from light to dark in its edge region. In addition, a retina protection stop can also be provided, which prevents illumination light from traveling into the interior of the patient's eye to the ocular fundus, where in some circumstances it could have a damaging effect. Close-to-axis illumination light is directed via a further field diaphragm of the illumination device into the object region, this further field diaphragm being imaged via a corresponding illumination optic into a second image plane in the object region. The latter plane is located at the retina of the patient's eye. A homogeneous and high-contrast red reflex at the patient's eye is said to be achieved with the corresponding surgical microscope; in the case of stereoscopic observation beam paths, it is favorable to make available a close-to-axis illumination beam path that is guided to the object region with multiple optical axes that correspond to the optical axes of the observation beam paths.

U.S. Pat. No. 5,627,613 A presents an ophthalmic illumination unit for a microscope which makes possible, thanks to corresponding placement of semi-transparent deflection elements and fully reflective deflection elements, illumination of a patient's eye at multiple illumination angles that are also adjustable, only one light source being used. This is intended in particular to protect the macula from damaging radiation. The corresponding angle of incidence can be modified by translational displacement of a deflection element. In this document as well, a close-to-axis and an off-axis illumination are implemented. In addition to the illumination beam paths guided through the main objective of the microscope, an even farther off-axis illumination can be implemented by way of an illumination beam path guided past the microscope objective.

In the case of the illumination device for a surgical microscope in accordance with DE 40 28 605 A1, illumination light is directed via the main objective of the microscope to the object point (patient's eye) at an angle of inclination of 6° to the optical axis (close-to-axis oblique illumination) by means of a first deflection element, and at an angle of inclination with respect to the optical axis that is variable between 0° and 4° (perpendicular or closer-to-axis oblique illumination) by means of a second deflection element. For this, the first deflection mirror (for the 6° illumination) comprises a cutout through which the illumination light can strike the second deflection mirror (for the 0°-to-4° illumination). The first deflection mirror can be continuously covered by means of a diaphragm until only the second deflection mirror is active for generating the 0° illumination. The second deflection mirror possesses a corresponding diaphragm so that it too can be covered in order to allow only the first deflection mirror for the 6° illumination to be effective.

JP 10133122 (A), publication date May 22, 1998, of Nippon, discloses an ophthalmic surgical microscope having the capability of switching between a coaxial illumination and a 6° illumination, or using both illumination types in combination. For this, a first prism for the coaxial illumination, which directs illumination light via the main objective onto the object, is arranged between the two observation beam paths of the stereomicroscope. Arranged parallel to the stereo base is a carriage on which a total-reflection prism and a semi-transparent prism are arranged, such that one of these two prisms can be brought into the illumination beam path by a motion of the carriage parallel to the stereo base. The latter prisms generate a 6° illumination. If the total-reflection prism is brought into the illumination beam path, a 6° illumination without coaxial illumination is implemented; if the semi-transparent prism is brought into the illumination beam path, a combined 6° and coaxial illumination is implemented; lastly, if neither of the two prisms is brought into the illumination beam path, exclusively coaxial illumination is then implemented.

The Applicant's German patent DE 10 2006 050 846 B4 describes a stereomicroscope in particular for ophthalmic surgery and neurosurgery, having a first and a second principal observer's beam path, which comprises an optical beam splitter device for generating an assistant's beam path and a documentation beam path. The versatile configurations of the optical beam splitter device that are described therein are intended to create an assistant's beam path and a documentation beam path, a simple assistant changeover being possible without unfavorably influencing the brightness conditions of the stereoscopic principal observation system in the context of such an assistant changeover. The assistant, or additional observer, can be placed selectably on the left or the right side of the microscope (and of the principal observer) without requiring the assistant's tube to be reinstalled or requiring two fixed assistant's tubes to be provided. The aforesaid document proposes for this purpose that in a first position of the beam splitter device, the assistant's beam path is outcoupled from the first principal observer's beam path, and in a second position of the beam splitter device is outcoupled from the second principal observer's beam path, such that the documentation beam path can be outcoupled from the respective other principal observer's beam path. The direction of the assistant's beam path in the first position is rotated 180° with respect to the direction thereof in the second position, and in both positions the outcoupled documentation beam path is respectively perpendicular to the outcoupled assistant's beam path.

Lastly, the Applicant's DE 10 2008 001 352 B4 discloses a stereomicroscope for use in ophthalmic or neurosurgical procedures, which (similarly to the stereomicroscope according to DE 10 2006 050 846 B4) possesses stereoscopic principal observer's beam paths and an optical beam splitter device for generating an assistant's beam path and a documentation beam path, in which microscope a simple assistant changeover is possible. In contrast to the previously recited document, the beam splitter device is embodied in such a way that in the first and in the second position of the beam splitter device, the assistant's beam path can be respectively outcoupled from the first main beam path, while the documentation beam path can be respectively outcoupled from the second main beam path. The number of moving parts in the beam splitter device can thereby be kept low.

### SUMMARY OF THE INVENTION

The object of the present invention is to describe a microscope, in particular a surgical microscope, furthermore in particular for ophthalmic procedures, that can be operated with outcoupling of a documentation beam path, such that the image quality for the principal observer and the documentation quality can be matched to one another. It is intended in particular that the microscope be capable of being operated with various illumination types, in particular close-to-axis and off-axis illumination, the intention being that matching of the image quality for the principal observer and/or the documentation quality can occur as a function of the respective illumination type.

The aforesaid object is achieved by a microscope of the kind mentioned previously, in which the beam splitter device in a first position outcouples the documentation beam path from the principal observer's beam path, and in a second position outcouples the documentation beam path from the assistant's beam path. The first and the second position of the beam splitter device are correlated here in particular with a first and second illumination type, respectively, of an illumination device of the microscope. In particular in the case of ophthalmic surgical microscopes, the first illumination type can be the close-to-axis illumination mentioned in the introduction to the specification by means of a first illumination beam path, and the second illumination type can be the aforementioned off-axis illumination by means of a second illumination beam path.

The invention further relates to a microscope having a principal observer's beam path and an assistant's beam path, the microscope being embodied as an ophthalmic surgical microscope and comprising an illumination device for generating a first and/or a second illumination beam path, said illumination device comprising, for generating the first illumination beam path, a deflection element that in its working position directs the first illumination beam path toward the object, the deflection element being configured pivotably respectively into its working position and out of its working position. The pivotably configured deflection element allows the image quality for the principal observer to be adjusted as a function of the illumination type. The first and the second illumination type are once again, in particular, close-to-axis and off-axis illumination, respectively. A documentation beam path does not obligatorily need to be provided with this type of microscope; when a documentation beam path is outcoupled, however, this microscope can, in accordance with a second variant, be additionally configured like the microscope in accordance with a first variant of the invention, i.e. can comprise a beam splitter device that outcouples the documentation beam path in a first position from the principal observer's beam path and in the second position from the assistant's beam path, the first and the second position of the beam splitter device being in particular once again correlated respectively with the first and the second illumination type.

Advantageous embodiments of the invention in accordance with the first and second variant are evident from the description that follows.

The microscope will first be described in accordance with a first variant of the invention. Microscopes of this type are known in particular as surgical microscopes, in particular ophthalmic surgical microscopes, as discussed in the introduction to the specification. A microscope of this kind possesses at least one principal observer's beam path and at least one assistant's beam path, and as a rule an optical beam splitter device for generating a documentation beam path. According to the present invention, the beam splitter device is configured in such a way that it can be switched between a first and a second position, such that in the first position of the beam splitter device, the documentation beam path is outcoupled from the principal observer's beam path. What this means is that in the case of exactly one principal observer's beam path, outcoupling occurs from that same beam path, while in the case of two stereoscopic principal observer's beam paths, outcoupling occurs in particular from one of the two beam paths. A monoscopic image is sufficient for documentation, so that outcoupling from exactly one beam path is sufficient. In the second position of the beam splitter device, outcoupling of the documentation beam path occurs from the assistant's beam path. If only one assistant's beam path is present, outcoupling thus occurs from that specific beam path; if two stereoscopic assistant's beam paths are present, outcoupling then occurs in particular from exactly one of the two assistant's beam paths.

The invention thus makes it possible on the one hand, when the beam splitter device is in the first position, to document exactly what the principal observer (surgeon) is observing. In the case of a surgical microscope, greater and greater value is being placed on exact documentation of surgical procedures. In the context of operations in the anterior segment of the eye, such as cataract operations, a good red reflex illumination is very important in terms of high image quality for the principal observer (surgeon). The red reflex illumination is as a rule not visible to the assistant. This is the case especially when the beam path for the red reflex illumination is directed by means of a beam splitter toward the object via the principal observer's beam path or one of the principal observer's beam paths. To allow the principal observer's image perceived under red reflex illumination and imaged onto the documentation camera as well, the optical beam splitter device is brought into its first position. This does reduce the transmittance in the principal observation beam path, but at the same time an optimum documentation image can be generated.

In the second position of the beam splitter device, the documentation beam path is outcoupled from the assistant's beam path so that the principal observer (surgeon) does not suffer from any transmittance losses but instead can work with optimum image quality. In the case of ophthalmic surgical microscopes, this setting is significant in particular for operations in the posterior segment of the eye. Red reflex illumination is generally not necessary for such procedures, so that the image outcoupled for documentation purposes does not obligatorily need to be picked off from the beam path of the principal observer. It is moreover important with such procedures, in particular when only the off-axis “background” illumination is being used, to provide the principal observer (surgeon) with sufficient light.

The invention thus makes possible a switchable documentation interface, i.e. the relevant beam splitter device can be operated in a first and in a second position in order to allow selection between very good documentation quality and optimized image quality for the principal observer. In the case of an ophthalmic surgical microscope, the invention allows a choice between optimized transmittance for the principal observer in the context of operations in the posterior segment of the eye, and optimized documentation for operations in the anterior portion of the eye.

The invention is also suitable in principle for microscopes in which the assistant's beam path is in turn outcoupled from the main beam path. This is often the case in particular with two-beam microscopes in which two main beam paths are available to the principal observer. The assistant's beam path is outcoupled from a first main beam path. With these types of microscope, the documentation beam path has hitherto been outcoupled from the other, second main beam path. The invention provides, in the context of this type of microscope, for outcoupling the documentation beam path from the assistant's beam path (second position of the beam splitter device) in the “maximum transmittance for the principal observer” operating mode, and for outcoupling the documentation beam path from the other, second main beam path (first position of the beam splitter device) in the “optimum documentation quality” operating mode.

Two-beam microscopes in which the principal observer and the assistant each have a beam path available to them are in principle also conceivable. Because both the principal observer and the assistant can perceive only a monoscopic image in such a case, a variant of this kind is less suitable for surgical microscopes. Four-beam microscopes, in which both the principal observer and the assistant each have a stereoscopic beam path available to them, are better suited for this purpose. This allows both viewers to see stereoscopic images. In this embodiment a specific assistant's beam path, which will be referred to hereinafter as “the relevant assistant's beam path,” is selected from the two assistant's beam paths. In the same fashion, a specific beam path, which will be referred to hereinafter as “the relevant principal observer's beam path,” is selected from the two principal observer's beam paths. In the first position of the beam splitter device, the documentation beam path is outcoupled from one of the two, namely from the relevant principal observer's beam path, while in the second position of the beam splitter device the documentation beam path is outcoupled from the other of the two, namely from the relevant assistant's beam path.

The invention is preferably usable in (two- or four-beam) microscopes in which the assistant's and principal observer's beam path or paths extend from a microscope objective separately from one another, parallel to the main axis of the microscope objective.

With no limitation of generality, the optical beam splitter for outcoupling the documentation beam path is implemented in one of the two embodiments discussed below.

The beam splitter device can comprise one, preferably exactly one, beam splitter, which in the first position of the beam splitter device is introduced into the relevant principal observer's beam path, while in the second position of the beam splitter device said beam splitter is introduced into the relevant assistant's beam path. The beam splitter is thus mounted displaceably—in the case of assistant's and observer's beam paths extending in parallel, in a direction perpendicular to said beam paths—in such a way that it can be introduced selectably into the relevant principal observer's beam path or into the relevant assistant's beam path in such a way that the documentation beam path is outcoupled from that respective beam path.

In another embodiment, the beam splitter device comprises two beam splitters, which will be referred to hereinafter as the “first” and the “second” beam splitter. In the first position of the beam splitter device, the first beam splitter is introduced into the relevant principal observer's beam path while the second beam splitter is removed from the relevant assistant's beam path. The documentation beam path is thus outcoupled from the relevant principal observer's beam path. In the second position of the beam splitter device, conversely, the first beam splitter is removed from the relevant principal observer's beam path while the second beam splitter is introduced into the relevant assistant's beam path. The documentation beam path is then therefore outcoupled from the assistant's beam path.

Microscopes according to the present invention generally comprise, besides a microscope objective, a zoom system. It proves to be advantageous to arrange the optical beam splitter device for outcoupling the documentation beam path behind the zoom system as seen from the microscope objective, in particular directly behind it, i.e. behind the last lens element of the zoom system.

As already repeatedly mentioned, the invention is usable with particular advantage in ophthalmic surgical microscopes. Such microscopes generally possess an illumination device for generating a first and/or a second illumination beam path. The first illumination beam path is, without limitation of generality, a coaxial or close-to-axis illumination beam path with which, for example, red reflex illumination can be implemented, while the second illumination beam path is an off-axis beam path that is used for ocular fundus illumination in the context of ophthalmic surgical procedures. While the first illumination beam path is always used for operations in the anterior segment of the eye, and optionally additionally the second as well, for operations in the posterior segment of the eye it is possible in principle to dispense with the first illumination beam path.

With an ophthalmic surgical microscope of this kind it is useful if, in the context of generation and utilization of the first, in particular only of the first, illumination beam path (i.e. the first illumination beam path is actually incident onto the object (patient's eye)), the beam splitter device is in the first position, with the result that the documentation beam path is outcoupled from the principal observer's beam path. It is further useful if, in the context of generation and utilization of the second, in particular only of the second, illumination beam path (i.e. for operations in the posterior segment of the eye), the beam splitter device is in the second position, with the result that the documentation beam path is outcoupled from the assistant's beam path.

It may be useful in general, in the context of generation and utilization of the first and/or of the second illumination beam path, to be able to bring the beam splitter device selectably into the first or the second position. The reason is that selection can then occur in application-dependent fashion; for example, the first position is selected for operations in the anterior segment of the eye, while the second position of the beam splitter device is selected for operations in the posterior segment of the eye.

In its second variant, the invention relates to a microscope having a principal observer's beam path and an assistant's beam path, the microscope being embodied as an ophthalmic surgical microscope and comprising an illumination device for generating a first and/or a second illumination beam path, the illumination device comprising, for generating the first illumination beam path, a deflection element (in particular, a semi-transparent mirror) that, in its working position, directs the first illumination beam path (in particular, through the microscope objective) toward the object (patient's eye), the deflection element being configured pivotably respectively into its working position and out of its working position. When the deflection element is pivoted out of its working position, the first illumination beam path then does not arrive at the object.

This variant of the invention is of particular significance, in a context of semi-transparent mirrors or prisms as deflection elements, if a semi-transparent deflection element of this kind is located in at least one principal observer's beam path in order to generate, as already explained above, a red reflex illumination. For operations under red reflex illumination, i.e. in particular operations in the anterior segment of the eye, the deflection element is pivoted into its working position. The first illumination beam path can then be used for coaxial or close-to-axis illumination. In addition, a second illumination beam path can be used for an off-axis “background” illumination. For operations in the posterior segment of the eye, conversely, the deflection mirror is pivoted out of its working position so that the first illumination beam path no longer strikes the object. Pivoting the deflection element out of its working position usefully causes it to be removed from the at least one principal observer's beam path, so that optimum transmittance exists therein. Pivoting of the deflection element out of the working position can also be performed when the illumination device is not generating the first illumination beam path, but instead only the second illumination beam path is being worked with. With this variant of the invention the aforesaid deflection element can thus be pivotable respectively into and out of the working position as a function of the application (pivoted into the working position for anterior ocular segment operations; pivoted out of the working position for posterior ocular segment operations) or as a function of the illumination light that is available (pivoted into the working position when the first, coaxial or close-to-axis illumination beam path is present; pivoted out of the working position when the first illumination beam path is switched off).

The aforementioned second variant of the invention can be used with particularly great advantage in combination with the first variant of the invention. For this, the deflection element is pivoted into its working position when the beam splitter device is in its first position, while the deflection element is pivoted out of its working position when the beam splitter device is in the second position. The result is that the already elevated transmittance for the principal observer becomes even further elevated in the second position of the beam splitter device, when the deflection element is pivoted out of its working position and thus causes no transmittance losses in the relevant principal observer's beam path. Conversely, when the beam splitter device is in the first position, the deflection element can then be pivoted automatically into its working position in order to direct the first illumination beam path onto the object.

The invention is schematically depicted in the drawings on the basis of an exemplifying embodiment, and will be described in detail below with reference to the drawings.

### BRIEF DESCRIPTION OF THE DRAWINGS

FIG. 1 schematically shows an exemplifying embodiment of a microscope according to the present invention having a beam splitter device, in a first embodiment;

FIG. 2 schematically shows a further exemplifying embodiment of a microscope according to the present invention having a beam splitter device, in a second embodiment; and

FIG. 3 shows another variant of a microscope according to the present invention.

### DETAILED DESCRIPTION OF THE INVENTION

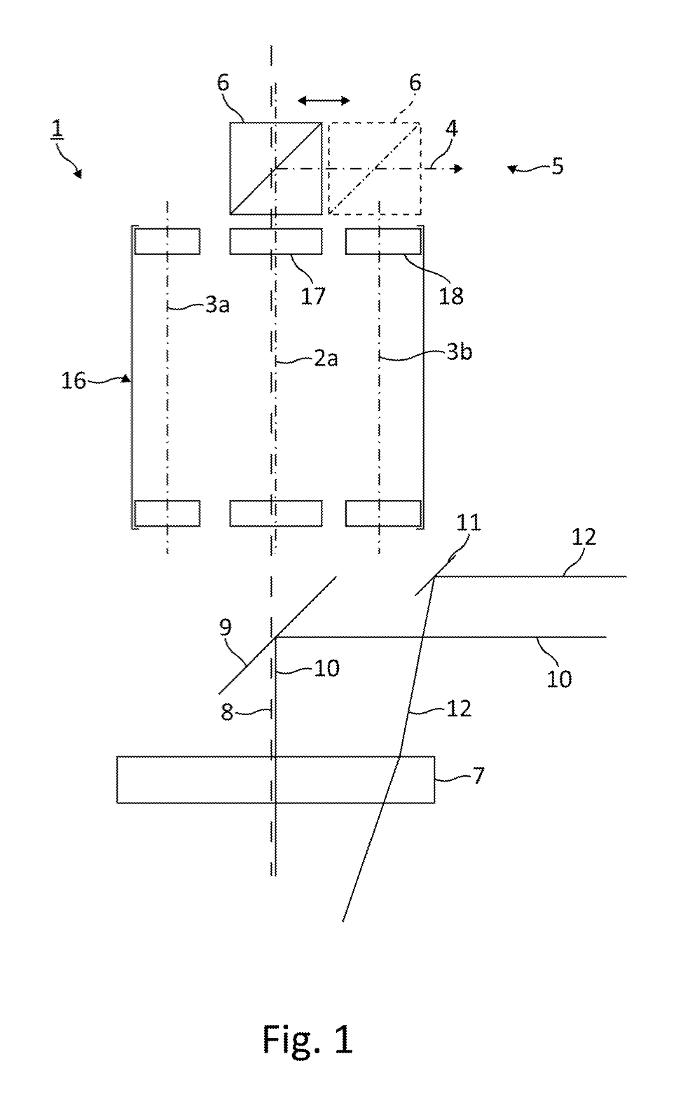

FIG. 1 shows a first embodiment of a microscope according to the present invention, in accordance with the first variant described above. FIG. 1 depicts only those parts of a microscope 1 that are essential for the invention. Microscope 1 encompasses a microscope objective 7 and a zoom system 16. The main axis of microscope objective 7 is labeled 8. The embodiment depicted refers to a four-beam microscope that provides both two principal observer's beam paths for the principal observer and two assistant's beam paths 3a and 3b for the assistant. The second (not depicted) principal observer's beam path is located behind the first principal observer's beam path 2a. In addition, for the sake of clarity only the respective axes of beam paths 2a, 3a, and 3b are depicted. Zoom system 16 encompasses multiple lens groups, only the first and last lens groups (as seen from objective 7) being depicted in the present case. Such zoom systems are known per se from the existing art, and will therefore not be further explained here. Directly adjacent to zoom system 16, more precisely directly behind the last zoom lens 17 of the relevant principal observer's beam path and the last zoom lens 18 of the relevant assistant's beam path, is a beam splitter device 5. A binocular tube, through which the principal observer (surgeon) can stereoscopically view the object being investigated (not depicted), is not shown. The same applies to the assistant's tube, which in this embodiment is likewise embodied as a binocular tube. Also known in addition to the telescope-type (Galileo system) stereomicroscope depicted here, which provides one common main objective for both principal observer's and assistant's beam paths, are stereomicroscopes of the Greenough type, in which a separate objective is provided for each principal observer's beam path. The invention is not limited to specific microscope types.

The channels (a total of four being present here) of zoom system 16 define the two principal observer's beam paths, of which only the front beam path 2a is depicted, as well as the two assistant's beam paths 3a and 3b. Instead of a zoom system 16, a magnification changer can in principle also be present. Also not depicted is a connector for a documentation module for (electro-optical) documentation of object observation. A documentation beam path 4 is outcoupled for this purpose.

With no limitation of generality, the discussion below will continue to be based on an ophthalmic surgical microscope having an illumination device (not depicted) that generates a first illumination beam path 10 and/or a second illumination beam path 12. First illumination beam path 10 is directed by a first deflection element 9 toward objective 7, from which the illumination beam path strikes the object. The case illustrated is that of coaxial illumination, in which first illumination beam path 10 proceeds coaxially with principal observer's beam path 2a. First deflection element 9 is accordingly embodied as a semi-transparent mirror. A further, second deflection element 11 directs second illumination beam path 12 toward objective 7, from whence it likewise strikes the object. Whereas a coaxial or close-to-axis illumination can be implemented with first deflection element 9, deflection element 11 is suitable for an off-axis illumination. As already stated repeatedly, the close-to-axis/coaxial illumination is suitable for operations in the anterior segment of the eye, while the off-axis kind is particularly well suited for operations in the posterior segment of the eye. This does not, however, preclude additionally directing second illumination beam path 12 onto the object in the case of procedures in the anterior segment of the eye as well. Nor is it precluded to direct first illumination beam path 10 onto the object in the context of operations in the posterior segment of the eye.

Following these general explanations, which are intended to be valid for all the FIGS. 1 to 3, beam splitter device 5 according to FIG. 1 will now be described in further detail.

In the embodiment depicted, beam splitter device 5 comprises only one beam splitter 6, which is mounted displaceably (indicated by the double arrow) between principal observer's beam path 2a and assistant's beam path 3b. The displacement direction extends perpendicularly to the axes of the relevant principal observer's beam path or assistant's beam path 2a, 3b. The first position of the optical beam splitter device 5 is depicted with solid lines, while the second position of beam splitter device 5 is indicated with dashed lines. Correspondingly, in the first position the beam splitter 6 is located in principal observer's beam path 2a and outcouples documentation beam path 4 from there. In the second position of beam splitter device 5, said beam splitter 6 is located in assistant's beam path 3b and outcouples documentation beam path 4 from there. In the first illumination type (coaxial illumination), a red reflex illumination is implemented by means of first illumination beam path 10. In this case beam splitter device 5 is in its first position, so that documentation beam path 4 contains exactly the same information as principal observer's beam path 2a from which documentation beam path 4 is being outcoupled. Optimum documentation quality of the surgical procedure is thus guaranteed in this position.

For operations in the posterior segment of the eye, in which first illumination beam path 10 often is not generated or is blocked out, beam splitter device 5 is in the second position (depicted with dashed lines) so that documentation beam path 4 is outcoupled from assistant's beam path 3b. Because the red reflex is usually not necessary for such procedures, documentation beam path 4 can also be outcoupled from assistant's beam path 3b without disadvantage, and with the advantage that principal observer's beam path 2a does not experience any transmittance loss.

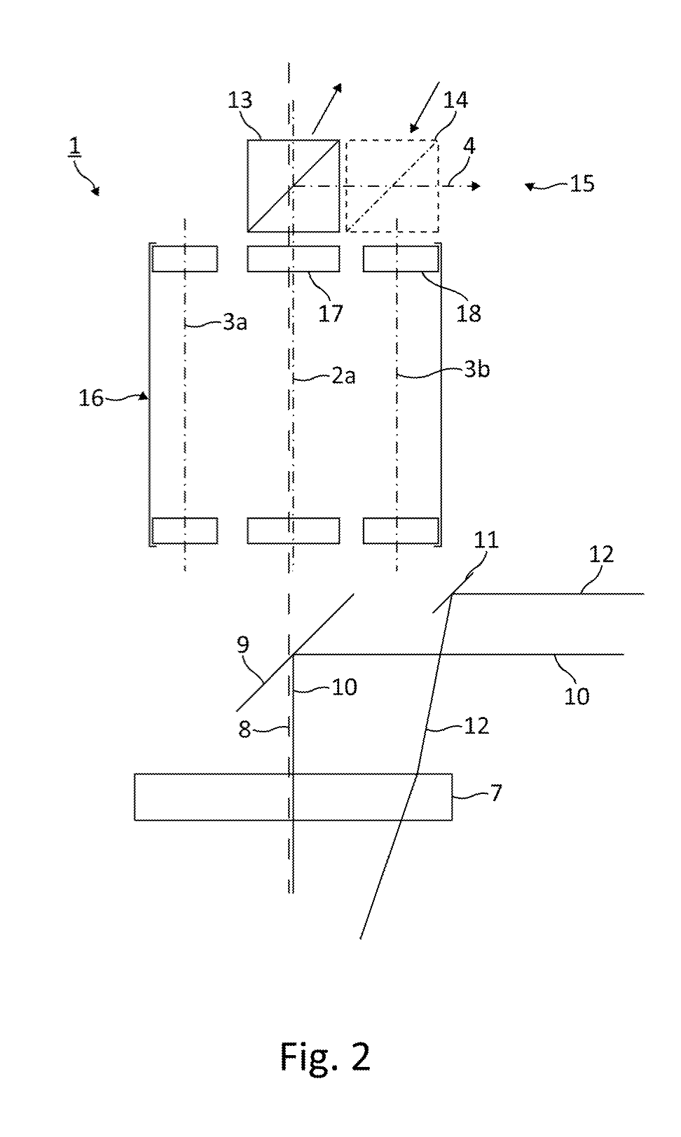

FIG. 2 shows a further embodiment of a microscope having a beam splitter device 15, in another configuration. Identical elements are provided with identical reference characters. Reference is made to the statements about FIG. 1 with regard to general explanations. Beam splitter device 15 now comprises two beam splitters, namely a first beam splitter 13 and a second beam splitter 14. Both principal observer's beam path 2a and assistant's beam path 3b therefore each have one beam splitter, both of which are pivotable into and out of the respective beam paths. In the first position of beam splitter device 15, first beam splitter 13 is thus located in principal observer's beam path 2a, while second beam splitter 14 is in a position pivoted out of assistant's beam path 3b, i.e. outside the effective cross section of assistant's beam path 3b. In the second position of beam splitter device 15, first beam splitter 13 is located outside the effective cross section of principal observer's beam path 2a, while second beam splitter 14 is located in assistant's beam path 3b. For further explanations of the first and the second position of beam splitter device 15 for the application instance of an ophthalmic surgical microscope, reference is made to the statements above.

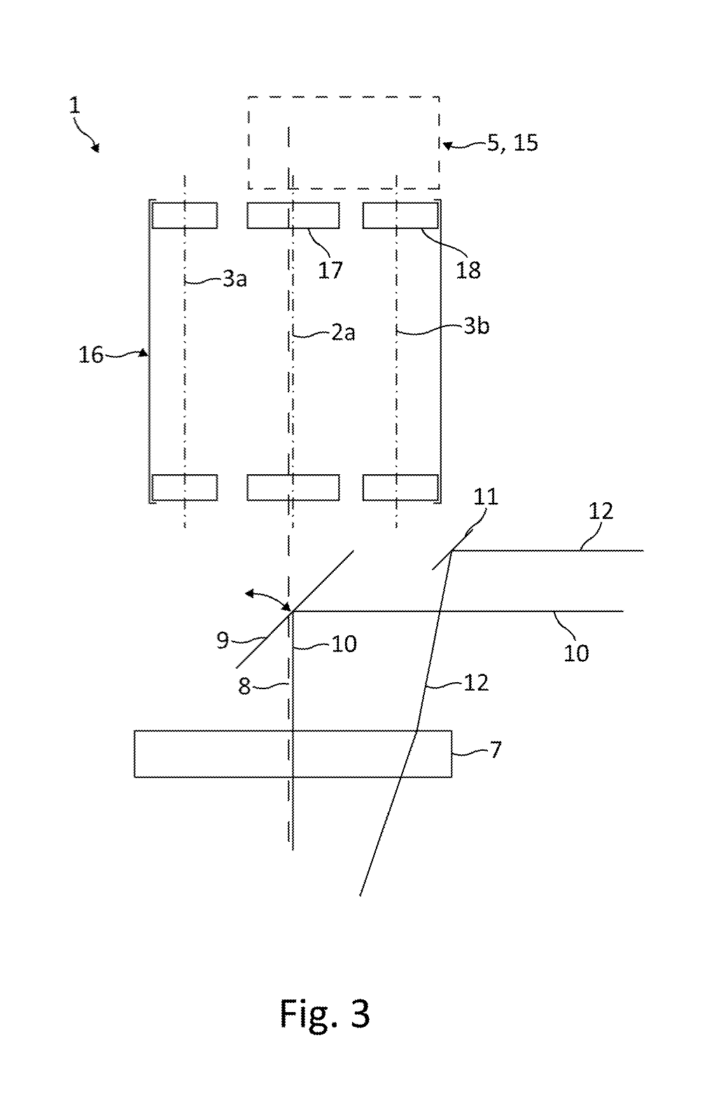

Lastly, FIG. 3 very schematically shows a microscope in another variant, in which first deflection element 9 is mounted pivotably (indicated by the double arrow). Microscope 1 depicted in FIG. 3 can preferably be equipped with a beam splitter device 5 according to FIG. 1, or with a beam splitter device 15 according to FIG. 2. Deflection element 9 is mounted pivotably; in the working position (depicted in FIG. 3) of deflection element 9, the latter is arranged in principal observer's beam path 2a in order to overlay first illumination beam path 10 coaxially onto principal observer's beam path 2a. Additionally or alternatively, a second illumination beam path 12 can be directed via deflection element 11 onto the object. In that regard, reference may be made once again to the statements above with regard to FIGS. 1 and 2.

First deflection element 9 is pivotable out of its working position; it is then no longer located in the effective cross section of principal observer's beam path 2a, so that the latter is opened up without transmittance losses.

First deflection element 9, which is embodied as a semi-transparent mirror, can thus also be automatically pivoted into or out of its working position as a function of the utilization of surgical microscope 1 or as a function of the illumination type. For operations in the anterior segment of the eye (first illumination beam path 10 is active) deflection mirror 9 is pivoted into its working position, while for operations in the posterior segment of the eye (second illumination beam path 12 is active) it is pivoted out of its working position. Deflection element 9 can furthermore be pivoted out of its working position when first illumination beam path 10 is deactivated or is to be deactivated.

A combination with a beam splitter device such as the one described in FIGS. 1 and 2 is particularly useful. When beam splitter device 5 or 15 is in its first position, deflection element 9 is pivoted into its working position. When beam splitter device 5 or 15 is in its second position, conversely, the deflection element is pivoted out of its working position in order to make even higher transmittance available to the principal observer.

The invention is not to be limited to the specific embodiments disclosed, and modifications and other embodiments are intended to be included within the scope of the invention.

### PARTS LIST

1 Microscope

2a Principal observer's beam path

3a, 3b Assistant's beam path

4 Documentation beam path

5 Beam splitter device

6 Beam splitter

7 Microscope objective

8 Main axis of objective

9 First deflection element

10 First illumination beam path

11 Second deflection element

12 Second illumination beam path

13 First beam splitter

14 Second beam splitter

15 Beam splitter device

16 Zoom system

17 Last zoom lens (principal observer)

18 Last zoom lens (assistant)

What is claimed is:

1. A microscope (1), comprising:
a principal observer's beam path (2a);
an assistant's beam path (3a, 3b); and
an optical beam splitter device (5, 15) for generating a documentation beam path (4);
wherein, in a first position, the optical beam splitter device (5, 15) is configured to outcouple the documentation beam path from the principal observer's beam path (2a), and in a second position configured to outcouple the documentation beam path from the assistant's beam path (3b).

2. The microscope according to claim 1, wherein the assistant's beam path (3a, 3b) and the principal observer's beam path (2a) extend from a microscope objective (7) separately from one another, parallel to the main axis (8) of the microscope objective (7).

3. The microscope according to claim 1, wherein the principal observer's beam path includes two stereoscopic beam paths (2a), wherein in the first position of the beam splitter device (5, 15) the documentation beam path (4) is configured to be outcoupled from one of the two principal observer's beam paths (2a).

4. The microscope according to claim 1, wherein the assistant's beam path includes two stereoscopic assistant's beam paths (3a, 3b), wherein in the second position of the beam splitter device (5, 15) the documentation beam path (4) is configured to be outcoupled from one of the two assistant's beam paths (3b).

5. The microscope according claim 1, wherein the beam splitter device (5) includes, for outcoupling the documentation beam path (4), a beam splitter (6) that in the first position of the beam splitter device is introduced into the principal observer's beam path (2a), and in the second position of the beam splitter device is introduced into the assistant's beam path (3b).

6. The microscope according to claim 1, wherein the beam splitter device (15) includes, for outcoupling the documentation beam path (4), a first beam splitter (13) and a second beam splitter (14), wherein in the first position of the beam splitter device the first beam splitter (13) is introduced into the principal observer's beam path (2a) and the second beam splitter (14) is removed from the assistant's beam path (3b), and wherein in the second position of the beam splitter device the first beam splitter (13) is removed from the principal observer's beam path (2a) and the second beam splitter (14) is introduced into the assistant's beam path (3b).

7. The microscope according to claim 1, further comprising a microscope objective (7) and a zoom system (16), wherein the optical beam splitter device (5, 15) is arranged behind the zoom system (16) as seen from the microscope objective (7).

8. The microscope according to claim 1, wherein the microscope is an ophthalmic surgical microscope having an illumination device for generating a first and/or a second illumination beam path (10, 12).

9. The microscope according to claim 8, wherein upon generation of the first illumination beam path (10), the beam splitter device (5, 15) is in the first position.

10. The microscope according to claim 8, wherein upon generation of the second illumination beam path (12), the beam splitter device (5, 15) is in the second position.

11. The microscope according to claim 8, wherein upon generation of the first and of the second illumination beam path (10, 12), the beam splitter device (5, 15) is selectably placeable into the first or the second position.

12. A microscope, comprising:
a principal observer's beam path (2a);
an assistant's beam path (3a, 3b); and
an illumination device for generating a first and/or a second illumination beam path (10, 12), the illumination device including, for generating the first illumination beam path (10), a deflection element (9) that in a working position directs the first illumination beam path (10) toward an object to be observed, the deflection element (9) being configured pivotably respectively into and out of the working position;
wherein the microscope is an ophthalmic surgical microscope.

13. The microscope according to claim 12, wherein the deflection element (9) is semi-transparent, and in the working position is located at least in part in the principal observer's beam path (2a).

14. The microscope according to claim 12, further comprising an optical beam splitter device (5, 15) for generating a documentation beam path (4);
wherein, in a first position, the optical beam splitter device (5, 15) is configured to outcouple the documentation beam path from the principal observer's beam path (2a), and in a second position configured to outcouple the documentation beam path from the assistant's beam path (3b)
wherein the deflection element (9) is pivoted into the working position when the beam splitter device (5, 15) is located in the first position.

15. The microscope according to claim 12, further comprising an optical beam splitter device (5, 15) for generating a documentation beam path (4);
wherein, in a first position, the optical beam splitter device (5, 15) is configured to outcouple the documentation beam path from the principal observer's beam path (2a), and in a second position configured to outcouple the documentation beam path from the assistant's beam path (3b);
wherein the deflection element (9) is pivoted out of its working position when the beam splitter device (5, 15) is located in the second position.
